(19) United States
(12) Patent Application Publication
(10) Pub. No.: US 2020/0155298 A1
(43) Pub. Date: May 21, 2020

(54) FIXTURES

(71) Applicant: Helix Fixation, Inc., Pleasanton, CA (US)

(72) Inventor: John F. KRUMME, Bainbridge Island, WA (US)

(21) Appl. No.: 16/687,302

(22) Filed: Nov. 18, 2019

Related U.S. Application Data
(60) Provisional application No. 62/768,896, filed on Nov. 17, 2018.

(51) Int. Cl. A61F 2/08

(52) U.S. Cl. CPC A61F 2/0811; A61F 2002/0888; A61B 2017/0414; A61F 2210/0023; A61F 2250/001; A61F 2002/0817

(57) ABSTRACT

An implant (fixture) device, systems, and methods for providing a fixture in, and/or around, and/or on, a substrate such a bone. The implant includes a hollow body composed of a shape-memory material. The hollow body includes a plurality of sections connected to each other and separated by an opening between the sections. The plurality of sections are configured to have a compact configuration and/or an expanded configuration and may change between the first configuration and the second configuration in response to an applied stimulus. The opening and the plurality of sections may be in the shape of a spiral.

### CROSS REFERENCE TO RELATED APPLICATIONS

This application claims priority to U.S. Provisional Patent Application No. 62/768,896 filed Nov. 17, 2018 and title “Fixtures” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates to implants or fixture devices for providing a fixture in, and/or around, and/or on, a substrate; to precursors for such fixture devices; to methods of making the fixture devices; to methods of making the precursors; to delivery systems for delivering the fixture devices; to methods of using the fixture devices; and to substrates including the fixture devices.

### BACKGROUND

Due to injury, disease, and aging, record numbers of surgeries are performed using implants placed in bones in a patient's body to secure tissue and aid in healing and recovery from a wide variety of damage. Bone implants are used extensively in open and minimally invasive surgery to reattach soft tissue to bone. The bone implant is secured to the bone and one or more sutures attached to the bone implant are used to secure the tissue to the bone. The tissue can be soft tissue anywhere in the body, e.g. a torn rotator cuff in a shoulder or a torn ligament. With over 3 million cases of rotator cuff tears and over 200,000 anterior cruciate ligament tears in the knee every year in the United States alone which are not adequately addressed by currently available techniques there is a need for improved bone implant devices, systems, and methods that are cost-effective, easy to use, and improve recovery. Provided herein are bone implant devices, systems, and methods that are cost-effective, easy to use, and improve recovery.

### SUMMARY OF THE DISCLOSURE

Described herein are implant devices (also called fixture devices) for providing an implant in, and/or around, and/or on, a substrate; to precursors for such implant devices; to methods of making the implant devices; to methods of making the precursors; to delivery systems for delivering the implant devices; to methods of using the implant devices; and to substrates including the implant devices. These implants can be placed in a human or other animal body. In one example, the fixture device is a bone anchor which is placed in a hole in a bone; and one or more sutures attached to the bone anchor are used to secure the bone to another part of the body, for example to tissue anywhere in the body (e.g. a torn rotator cuff in a shoulder, or a torn ligament), or to another bone, or to a bone fragment. In other examples, the fixture device is a compression screw, a plate anchoring device, a syndesmosis device, a tendenodesis device or a device for broken bone reduction.

One aspect of the disclosure provides an implant including a hollow body composed of a shape-memory material, at least part of the hollow body including a plurality of sections which are connected to each other and which are in either (i) a first, compact configuration in which the sections are relatively close to each other or (ii) a second, radially expanded configuration in which the sections are relatively distant from each other and longitudinally separated by an opening through a wall of the hollow body. In some embodiments, the hollow body is configured to change between the first compact configuration and the second expanded configuration in response to an applied stimulus.

In some embodiments, the sections include a spiral including shape-memory material.

In some embodiments, the plurality of sections are in contact with one another when the hollow body is in the first, compact configuration. In some embodiments, the opening includes a spiral shaped opening. In some embodiments, the hollow body includes a cylindrical body.

Some embodiments include mechanical purchase points on the proximal and/or distal ends, wherein the implant is configured to change between the first compact configuration and the second expanded configuration in response to moving the mechanical purchase points relative to the hollow body. Some embodiments include mechanical purchase points on the proximal and/or distal ends configured to be held by an implant inserter device.

In some embodiments, the shape memory material includes a nickel titanium alloy. In some embodiments, the shape memory material includes a nickel titanium alloy containing about 55-56% by weight of nickel. In some embodiments, shape memory material includes a nickel titanium alloy containing about 44-45% by weight of titanium.

In some embodiments, the hollow body includes an orifice configured to pass a suture therethrough.

In some embodiments, the applied stimulus includes applying heat.

Another aspect of the disclosure provides a method of preparing an implant including expanding a plurality of sections in a hollow body composed of a shape-memory material, at least part of the hollow body including the plurality of sections which are connected to each other and which are in a first, compact configuration in which the sections are relatively close to each other to form a second, expanded configuration in which the sections are more distant from each other and separated from each other by an opening through a wall of the hollow body.

Some embodiments includes the further step of heat-setting the expanded plurality of sections with an application of heat. Some embodiments include the further step of deforming the implant to convert the plurality of sections into the first, compact configuration. In some embodiments, the deformed implant includes a shape-memory of the expanded configuration.

In some embodiments, the step of expanding includes rotating the proximal end and/or the distal end of the implant relative to a portion of the hollow body. In some embodiments, the step of expanding includes rotating the proximal end of the implant in a first direction while rotating the distal end in the opposite direction.

In some embodiments, the plurality of sections are separated by a spiral cut and the step of expanding includes expanding a height of the spiral cut. In some embodiments, the plurality of sections include a spiral having a diameter and the step of expanding includes expanding the diameter of the spiral.

Some embodiments further include the step of modifying a proximal end and/or a distal end of the implant with mechanical purchase points.

Another aspect of the disclosure provides a method of implanting an implant in a substrate including the step of placing an implant including a proximal end, a distal end, and a hollow body therebetween into a hole in a substrate, the hollow body including a shape-memory material, at least part of the hollow body including a plurality of sections which are connected to each other and which are in a first, compact configuration in which the sections are relatively close to each other and separated by an opening through a wall of the hollow body. Some embodiments include the step of expanding with an application of heat the plurality of sections to form a second, expanded configuration in which the opening is larger and the sections are more distant from each other.

In some embodiments, the step of expanding includes rotating the proximal end and/or the distal end of the implant relative to hollow body. In some embodiments, the step of expanding includes rotating the proximal end of the implant relative to the distal end. In some embodiments, the step of expanding includes rotating the proximal end and distal end of the implant in opposite directions.

In some embodiments, the step of expanding includes contacting an outer surface of the wall with the substrate. In some embodiments, the step of expanding includes contacting mechanical purchase points on the proximal end and/or distal end of the implant with an inserter device.

In some embodiments, the application of heat includes heat from a mammalian body.

In some embodiments, the sections are between 0.5 mm and 5 mm from each other in the second, expanded configuration.

In some embodiments, the shape memory material includes a nickel-titanium alloy. In some embodiments, the shape memory material includes a nickel titanium alloy containing about 55-56% by weight of nickel. In some embodiments, the shape memory material includes a nickel titanium alloy containing about 44-45% by weight of titanium.

Some embodiments include the additional step of attaching a suture between the implant and a soft tissue in a patient. In some embodiments, the substrate includes a bone, and the method further includes the step of drilling a hole in the bone.

Another aspect of the disclosure provides a method of making an implant including making a cut through the wall of a central section of a hollow body, the wall composed of a shape-memory material and forming a plurality of sections which are connected to each other and separated by the cut, wherein the plurality of sections are relatively close to each other to form an implant in a first compact configuration.

In some embodiments, the cut is a spiral cut made in the central portion of a tube of constant annular section. In some embodiments, the spiral cut includes at least one turn.

In some embodiments, the shape memory material includes a nickel titanium alloy. In some embodiments, the shape memory material includes a nickel titanium alloy containing about 55-56% by weight of nickel. In some embodiments, the shape memory material includes a nickel titanium alloy containing about 44-45% by weight of titanium.

Some embodiments further include the step of making a second cut through the wall of the central section of a hollow body and forming a second plurality of sections which are connected to each other.

Another aspect of the disclosure provides a method of making an implant including making a first cut through the wall of a central section of a hollow body, the wall composed of a shape-memory material and making a second cut parallel to the first cut through the wall. Some embodiments include the step of removing wall material between the first cut and the second cut to form an opening. Some embodiments include the step of forming a plurality of sections which are connected to each other and separated by the cut, wherein the plurality of sections are in a second, expanded configuration in which the sections are relatively distant from each other and separated by an opening through a wall of the hollow body

In some embodiments, the cut is a spiral cut made in the central portion of a tube of constant annular section. In some embodiments, the spiral cut includes at least one turn. Some embodiments further include the step of making a second cut through the wall of the central section of a hollow body and forming a second plurality of sections which are connected to each other.

In some embodiments, the shape memory material includes a nickel titanium alloy. In some embodiments, the shape memory material includes a nickel titanium alloy containing about 55-56% by weight of nickel and about 44-45% by weight of titanium.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view of an implant composed of a shape-memory material and a hollow cylinder with a spiral cut in the central portion.

FIG. 2 is a side view of the shape-memory implant shown in FIG. 1.

FIG. 3 is a perspective view of the shape-memory implant shown in FIG. 1 after the spiral cut has been opened to expand the central portion and the central portion has been heat set in the expanded configuration.

FIG. 4 is a perspective view of the shape-memory implant shown in FIG. 3 after collapsing the opened spiral cut to contract the expanded central portion. FIG. 4 also shows the implant inserted into a substrate.

FIG. 5 is a perspective view of the shape-memory implant shown in FIG. 4 after the spiral cut in the contracted central portion has been opened to expand the implant in the substrate. The wall of the implant spiral contacts the internal wall of the substrate.

FIG. 6A, FIG. 6B, FIG. 6C, and FIG. 6D show cross-sections of variations of different spiral cuts on shape-memory implants.

FIG. 7 shows an implant with different types of spiral openings.

FIG. 8A is a perspective view of a shape-memory implant with a spiral cut in which the coils of the spiral on the implant are separated and the sections are in an expanded configuration. FIG. 8B is a perspective view of the shape-memory implant shown in FIG. 8A after torqueing to bring the sections closer together. FIG. 8C is a perspective view of the implant shown in FIG. 8B after it has been inserted into a hole in a substrate and reverted to or towards the configuration shown in FIG. 8A.

FIG. 9A shows a delivery device for delivering an implant into a substrate. FIG. 9B shows a perspective view of the proximal end of a delivery device for delivering an implant into a substrate.

### DETAILED DESCRIPTION

Described herein are implantable fixture devices (implants) for providing a fixture in, and/or around, and/or on, a substrate; to precursors for such fixture devices; to methods of making fixture devices; to methods of making the precursors; to delivery systems for delivering the fixture devices; to methods of using the fixture devices; and to substrates including the fixture devices. In a variety of situations, it is desirable to provide a fixture in, and/or around, and/or on, a substrate, so that the substrate is linked, or can be linked, to another component, and/or so that the substrate is reinforced; to methods of using the implant devices; and to substrates including the implant devices. These implants can be placed in a human or other animal body. In one example, the fixture device is a bone anchor which is placed in a hole in a bone; and one or more sutures attached to the bone anchor are used to secure the bone to another part of the body, for example to tissue anywhere in the body (e.g. a torn rotator cuff in a shoulder, or a torn ligament), or to another bone, or to a bone fragment. In other examples, the fixture device is a compression screw, a plate anchoring device, a syndesmosis device, a tenodesis device or a device for broken bone reduction.

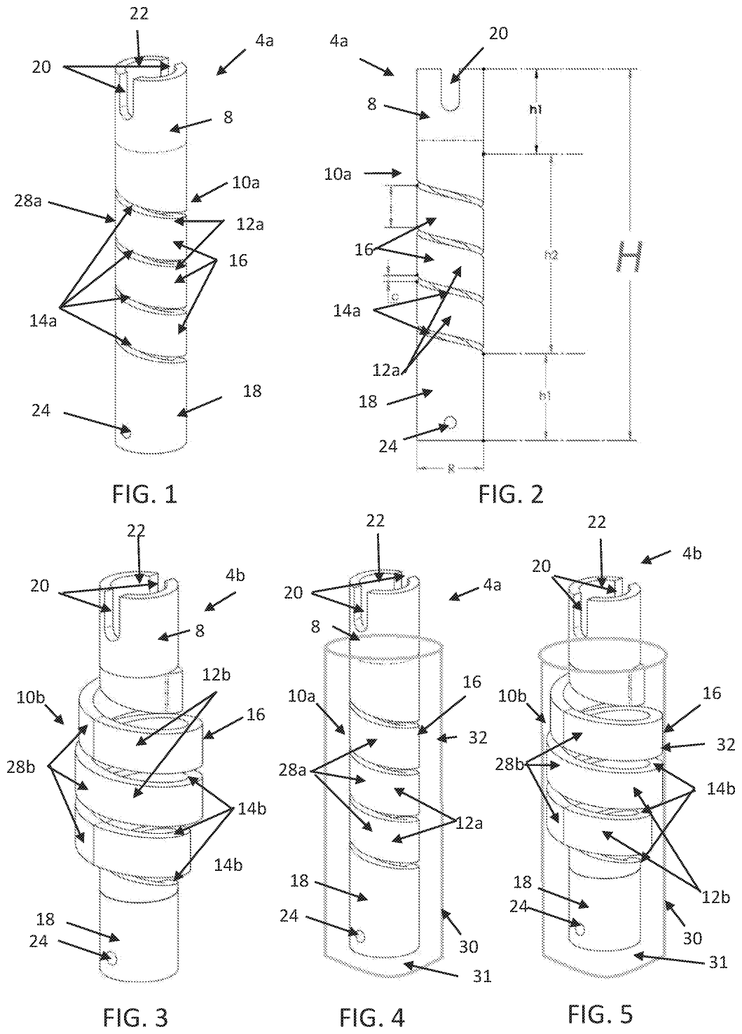

FIG. 1 is a perspective view of an implant 4a at least partially composed of a shape-memory material and that can be in a first contracted configuration or a second expanded configuration. The implant 4a may be configured to adopt the configuration it is not in (e.g., the first contracted or the second expanded) in response to a stimulus. FIG. 2 is a side view of the shape-memory implant 4a shown in FIG. 1. The implant 4a includes a hollow body (e.g., a cylinder) with a plurality of sections, including a proximal end 8, a distal end 18, and a compact central portion 10a between the proximal end 8 and the distal end 18. The implant 4a has an opening 14a in the form of a spiral cut in the compact middle portion 10a of the implant forming a spiral in the compact middle portion 10a. The spiral in the compact middle portion 10a includes a plurality of turns 12a. The spiral cut of the opening allows the implant 4a to expand and contract. In the first (compact) configuration the sections are relatively close to each other (including being in contact with each other) and in the second (expanded) configuration the sections are relatively distant from each other (e.g., further apart longitudinally than in the first (compact) configuration. In the expanded configuration of this embodiment, the plurality of turns 12b are both radially expanded and longitudinally expanded. The expansion and contraction may allow easier insertion of the implant 4a into a substrate in a first configuration and better contact between the implant and the substrate when the implant 4b is in the second configuration. The sections can be converted from the first compact configuration to the second expanded configuration, or vice versa, by any suitable procedure. The shape of components composed of a shape-memory material can be modified by suitable treatments at different temperatures. The procedure can include a step which includes heat setting one or more (including all) of the sections. In some variations, the hollow body includes a single spiral or two or more adjacent spirals which are composed of a shape-memory material. The procedure can include a step which includes mechanically setting one or more (including all) of the sections. The term “sections”, as used in this specification, includes different parts of a spiral.

FIG. 3 is a perspective view of the shape-memory implant shown in FIG. 1 and FIG. 2 after the spiral cut of the opening 14b has been expanded/opened to expand the contracted central portion 10a into the expanded central portion 10b and the expanded central portion 10b has been heat set in the expanded configuration. Use of heat to heat-set the shape of the implant at this stage allows implant to regain the heat-set shape after the implant is implanted into, onto, or around a substrate 30. The height of the spiral cut of the opening 14a in the compact central portion 10a is shown by c in FIG. 2. The height c of the spiral cut is expanded when the implant is in the second expanded configuration such as that shown in FIG. 3 and FIG. 5. The expanded central portion 10b may be expanded in length and/or diameter, and if the implant 4b is not cylindrical, the expanded central portion 10b may expand in none, either, or both lateral dimensions (e.g., relative to the axis of the hollow portion). The height of the turn of a spiral in the implant 4b is represented by the letter tin FIG. 2. The diameter R of the implant 4a in FIG. 2 is shown as constant. (In some variations, the diameter R may vary along the vertical axis). When the implant is in the expanded state, the diameter of the implant 4a in FIG. 2 increases over at least part of the implant which may over a part or all of the central portion of the implant. In some variations, the diameter shown as a constant in FIG. 2 is no longer constant over the length of the implant 4a when the implant in the expanded state. The proximal end 8 and the distal end 18 have a diameter of R₁ (not shown) while at least part of the expanded central portion 10b has a diameter R₂ (not shown). In this example, R₂>R₁. Furthermore, the diameter along the expanded central portion 10b may vary. In some variations, the diameter along the proximal and/or distal portions of the expanded central portion 10b may be smaller than the diameter of the middle part of the expanded central portion 10b (and there may be a gradient in the sizing. The implants described herein may be made from a shape-changing material, such as a shape-memory alloy. The shape-memory material may have properties of shape memory effect and superelasticity/pseudoelasticity. The implants may be made from a nickel-titanium alloy, for example an alloy containing 55-56% by weight nickel and the balance titanium, such as 55.8% titanium. The implants may be made from a nickel-titanium alloy which is 20% to 80% nickel, or 49% to 59% by weight nickel, such as 55-56% by weight nickel and the remainder titanium and/or another material. The implants may be made from 20% to 80% titanium, such as 40% to 55% titanium or 44%-45% titanium and the remainder nickel and/or another material. Other materials may include aluminum, copper, niobium, vanadium, and zinc such as a copper-aluminum material. An implant as described herein may include materials other than shape memory material, such as a biocompatible polymer such as a biocompatible fiber reinforced polymer or other materials. The implants (e.g., precursors and the fixtures) can include other components that are made of materials other than a shape-memory alloy, providing that such other components are composed of a material that can remain in the human body, and do not have a significant adverse impact on the performance of the implant. The implant 4a and implant 4b may be made of one or more non-shape memory materials or combinations thereof, such as chrome, cobalt, cobalt-chrome, gold, platinum, silver, iridium, stainless steel (e.g., surgical stainless steel), tantalum, titanium, or tungsten. The other materials may be integrally formed into an implant or placed (e.g., coated or sprayed) on a surface of the implant. In some variations, an implant may be monolithic (e.g. formed from a single piece of material and have no adhesive, glue, joints, nails, or screws holding it together) or a first portion of an implant may be made from a first material and a second portion of an implant may have an outer added material (e.g., a coating or spraying). In some variations, the proximal end 8 and the distal end 18 of the implant are made from different materials than the compact central portion 10a or expanded central portion 10b. The components described herein including a shape metal alloy can be modified by suitable treatments at different temperatures. One method of manufacturing the implant (e.g., the precursor) includes making cuts through the wall of at least the central section of a hollow body composed of a shape-memory material such as a shape-metal alloy. In some such methods, at least one spiral cut is made in the central portion of a tube of constant annular section. Another method of manufacturing the implant includes converting a strip of a shape-memory material (e.g., shape-memory metal such as TiNi) into a helix using conventional spring-winding technique while simultaneously heat setting the helix.

FIG. 4 is a perspective view of the shape-memory implant 4a shown in FIG. 3 after collapsing the opened spiral cut to contract the expanded central portion 10b into the compact central portion 10a. FIG. 4 also shows the implant 4a inserted into a substrate 30, prior to re-expansion of the implant 4a. The substrate 30 may be a bone hole, such as a space between a bone and another tissue (bone) or a hole drilled in a bone by a surgeon using a bone drill.

FIG. 5 is a perspective view of the shape-memory implant shown in FIG. 4 after the spiral cut in the contracted central portion 4a has been opened to expand the contracted central portion, such as using body heat (from the patient), applied heat, or a mechanical action to expand the implant in the substrate. The wall 28b of the implant 4b is in contact with the internal wall 32 of a hole 31 the substrate 30 and may hold the implant 4b in place such as using friction or a mechanical holding action. In some variations, a suture (not shown) attached to the implant 4b may be sutured to a soft tissue, such as a tendon or ligament outside the hole. A feature 24 may be configured to receive a suture. The internal diameter of at least part of the hole 31 in the substrate 30 may be smaller than the maximum diameter R of the implant 4b (e.g., as shown in the heat-set implant 4b shown in FIG. 3). The spiral in the implant 4b may exert an outward/expansive force on the inner wall 32 of the substrate 30 and hold the implant 4b in place. Over time and due to normal tissue mechanics, a substrate 30 such as a bone, may compress, decay, or resorb, and the outward/expansive force on the inner wall 32 by the implant 4b may continue to expand as the substrate 30 compresses, decays, or resorbs, and continue to hold implant 4b in place inside substrate 30. In the absence of the implants described herein and/or an outward/expansive force exerted by the implants described herein, an implant might otherwise loosen or move and not hold the tissue it was intended to hold. Such loosening or movement of an implant may cause a loss of functionality in a tissue or joint. In some cases, the implant may fail and the patient may require revision surgery, an additional surgery performed to replace or compensate for a failed implant.

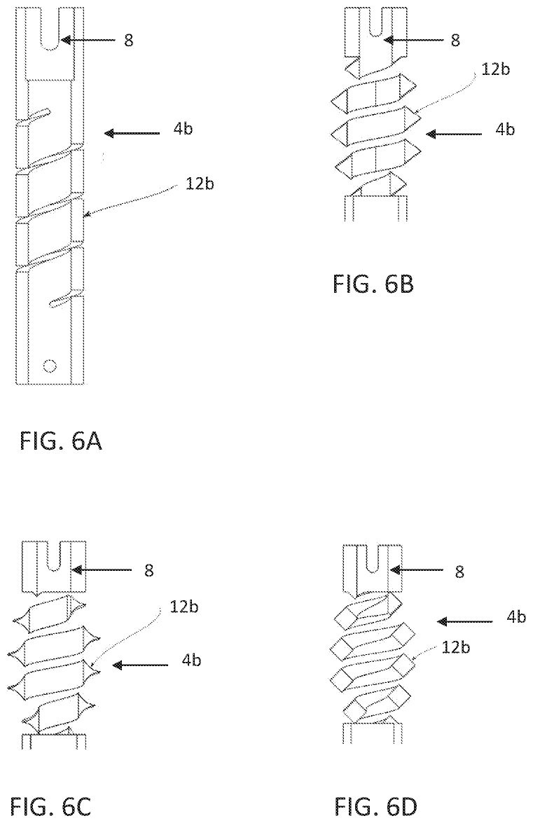

FIG. 6A shows a cross-section through the expanded implant 4b showing the inside of spiral turns on the implant. FIG. 6B, FIG. 6C, and FIG. 6D show other variations of the spiral portion, including triangular, saddled, and square, and the cross-section of the spiral turns may be either regular or irregular and may be bulging, curved, flattened, or rounded. Such geometrical features may aid in implant delivery to a particular body structure and/or aid in holding an implant in place (through additional friction or gripping).

Although FIGS. 1-5 show the implant as a hollow cylinder with a spiral cut for illustration purposes, the implant may have other structures and other shaped cuts. The implant 4a/implant 4b may be non-cylindrical, such as hexagonal or oval shaped (ovoid).

One method of making the implant (e.g., a precursor form of the implant) includes making cuts through the wall of the central section of the hollow body composed of a shape-memory material which is in the superelastic state. In some variations, at least one spiral cut is made in at least the central portion of a tube of constant annular section. The tube can for example have a length of up to 40 mm, e.g. 1-25 mm, a diameter of 1-25 mm, and a wall thickness of 0.1-2.5 mm. However, this method can be used with other generally tubular hollow bodies that include a shape-memory material. For example, the cross-section of the tube can be non-annular, e.g. oval or hexagonal; and/or the cross-section can change along the length of the tube, for example can change in diameter (or other dimension of width of a hollow body having a non-annular cross-section). In some variations, the one or more spiral cuts result in a hollow body in which all the sections are separated only by the width of the cut (which can be of the order of 0.005 mm for some cuts, such as when the cutting is made by laser). Thus, in this variation, the sections, when the precursor is first prepared, are in the compact configuration. As discussed below, in this variation, the preparation of the implant includes a step in which the compact sections are converted into the expanded configuration and heat set in the expanded configuration.

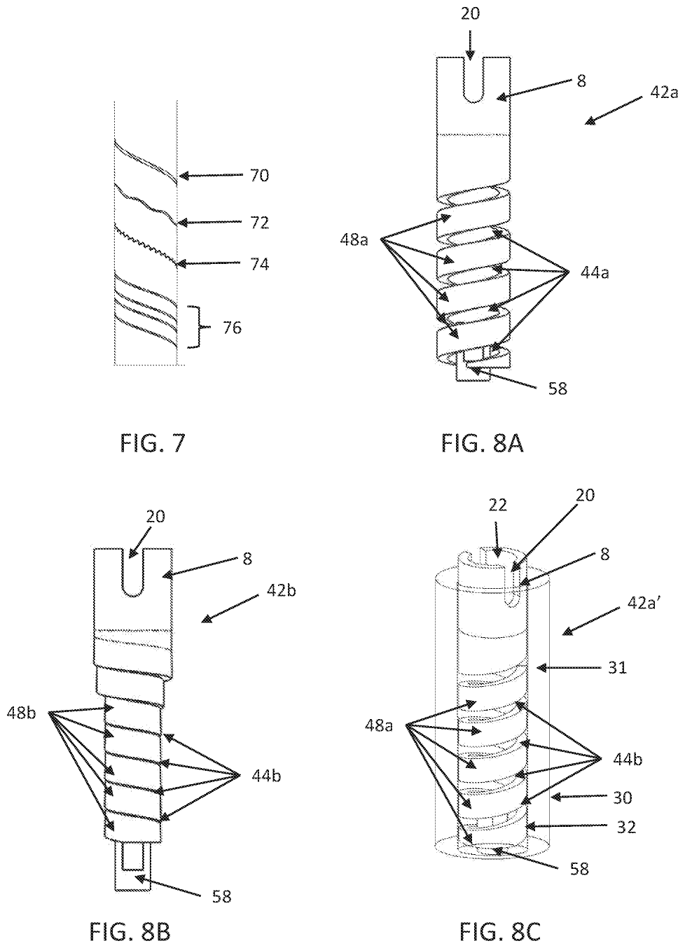

In some variations, two or more parallel spiral cuts are made to produce a hollow body in which the sections are separated from each other. Thus, in this variation, the sections, when the precursor is first prepared, are in the second expanded configuration (the shape-memory alloy between the spiral cuts is removed and discarded). The heights of the different sections can be the same (as shown for example in FIG. 7) or different. In this variation, the inclusion of a heat-setting step is optional. The height of the components of the spiral (i.e. the distance between the cuts when the precursor is prepared by cutting adjacent spiral cuts into a hollow body, which is the dimension designated “t” in FIG. 2) can be selected with a view to the proposed use of the device. For example, for a bone anchor, the dimension could for example be 0.5 mm-2.0 mm, and for a compression screw the dimension could for example be 2.0 mm-5.0 mm. The cutting of the hollow body can for example be made by means of a laser or a waterjet. More complex cuts may be more easily produced when using a laser cutter. The cross-section (viewed horizontally from the side) of the spirals can be selected, using a single laser beam or two or more laser beams set at different angles to the vertical. Examples of different cross-sections are shown in FIGS. 6A-6D. If the cross-section has a point on the outside of the spiral, this can increase the extent to which the expanded section penetrates the interior of a hole into which the fixture device is inserted. If the cross-section has a point on the inside of the spiral, this can increase the extent to which the compact sections penetrate the exterior of a substrate around which, or over which, the implant fixture device is applied. The shape and the slope of the spiral cut, viewed from the side, can also be selected. Examples of various shapes and slopes are shown in FIG. 7. For simplicity the spiral cut 70, wavy cut 72, zig-zag cut 74, and double spiral cut 76 are shown on a single implant though in practice, an implant may have only one or a combination of these cuts. Although shown as a continuous cut in FIG. 1, an implant may have two or more discontinuous cuts. FIG. 7 shows one of a series of consecutive spiral coils (the spiral cut 70, wavy cut 72, zig-zag cut 74, and double spiral cut 76) and these may be repeated above and below on an implant.

FIG. 8A is a perspective view of an implant 42a (a precursor) in which the coils of the spiral are separated, so that the sections 48a are in the expanded configuration (the parts of the tube which were between the coils have been removed e.g., by cutting). FIG. 8B is a perspective view of the implant 42b after the implant 42a in FIG. 8A has been torqued to bring the sections 48b close to each other. FIG. 8C is a perspective view of implant 42a′ after the implant shown in FIG. 8B has been inserted into the hole 31 in the substrate 30 and has reverted to or towards the configuration shown in FIG. 8A. The implant 42a′ shown in FIG. 8C that has not fully returned to the expanded configuration may exert an outward force on substrate 30 as it attempts to revert to the shape-memory configuration shown in FIG. 8A. The implant 42a may advantageously continue to exert the outward force for 1-24 hours, 1-30 days, 1-12 months, or years. Thus, as the substrate 30 resorbs, shrinks, or otherwise changes shape over time, the outward force urging the implant 42a′ to its expanded configuration continues to hold the implant 42a′ in place. It may prevent or minimize any implant slipping or moving out of a desired position. Despite resorption of bone, an all-too-common occurrence after injury or bone surgery, the implant 42a is more likely to stay firmly in place, and may hold any soft tissue, such as tendons or ligaments. The characteristics and methods described elsewhere herein for implants (such as for the implant shown in FIG. 1-FIG. 7) also apply here. For example, the implant may be made of a shape-memory alloy as described for FIG. 1-FIG. 7, the dimensions of the spiral and spiral cut described for FIG. 1-FIG. 7 apply here, etc. In some variations, the implant is a fixture device which is ready for delivery. The fixture device may be or may include a precursor. In some variations, the fixture device is suitable for delivery into or onto a substrate. The fixture device, as it is delivered, includes a precursor in which the sections are in a compact configuration but were previously heat set in an expanded configuration and thereafter deformed to convert the sections into the compact configuration. In this variation, the fixture device, after it has been delivered, is treated so that the sections are converted to or towards the heat-set configuration, and thus contact the substrate. In some variations, the fixture device is suitable for delivery into or onto a substrate. The fixture device, as it is delivered, includes a precursor in which the sections are maintained in an elastically deformed/compact configuration; and after the fixture device has been delivered, the compact sections are released from the elastically deformed compact configuration and become expanded sections that contact the substrate. In another variation, the fixture device is suitable for delivery around a substrate. The fixture device, as it is delivered comprises a precursor in which the sections are in an expanded configuration but were previously heat set in a compact configuration and thereafter deformed to convert the sections into the expanded configuration. In this variation the fixture device, after it has been delivered, is treated so that the sections are converted to or towards the heat set configuration, and thus recover around the substrate. In some variations, the fixture device is suitable for delivery around a substrate. The fixture device, as it is delivered, comprises a precursor in which the sections are maintained in an elastically deformed expanded configuration, and after the fixture device has been delivered, the expanded sections are released from the elastically deformed configuration and become compact sections that contact the substrate.

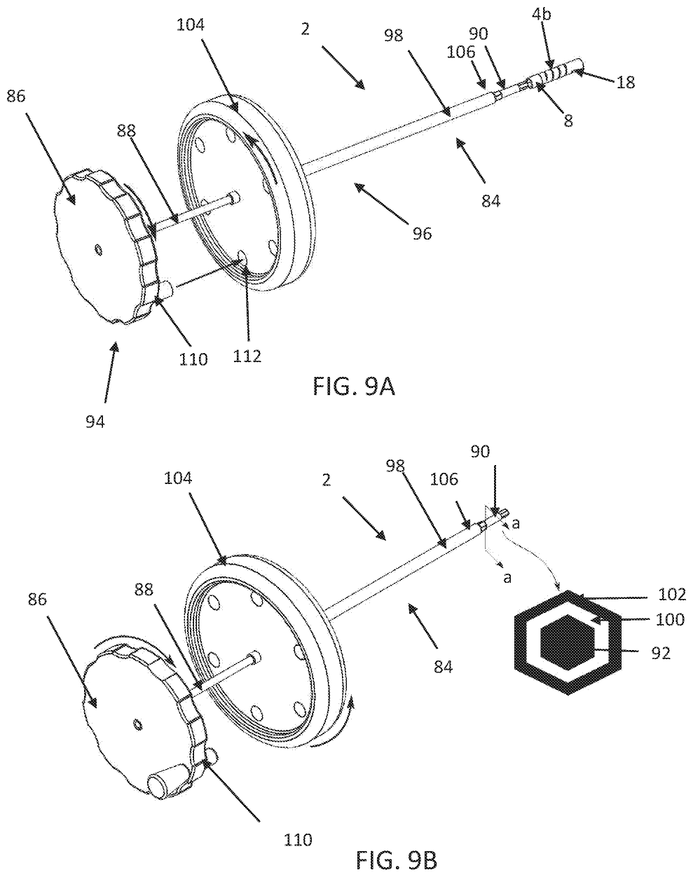

FIGS. 9A-9B show a delivery system 2 with an inserter tool 84 for inserting, rotating, torqueing, expanding, contracting, and/or removing an implant such as implant 4a or 4b. The inserter tool 84 has an outer tool 96 and an inner tool 94. The outer tool 96 has an elongate outer shaft 98 with a proximal wheel 104 and a distal end 106. The inner tool 94 has an elongate inner shaft 88 with a proximal knob 86 and a distal end 90. The elongate outer shaft 98 is configured to receive the elongate inner shaft 88 and the elongate inner shaft 88 extends distally past the distal end 106 of the elongate outer shaft 98. To deliver an implant 4b to a substrate 30, FIG. 9A shows the implant 4b is mounted on the distal end 106 of the elongate inner shaft 88 which extends through the hollow center 22 of implant 4b and engages the implant 4b at the distal end 18 of the implant 4b. The distal end 106 of the elongate outer shaft 98 engages the proximal end 8 of the implant 4b such as through a feature 20 configured to mate or be held by the elongate outer shaft 98. As shown in the cross-sectional view shown in FIG. 9B (and indicated by the cross-section arrow a-a proximal to the implant) hex 92 of the inner elongate shaft fits inside the hex 102 of the outer elongate shaft. The space 100 between allows the hex 92 and the hex 102 to rotate independently (when the proximal ends of the inner and outer elongate shafts are not attached to each other). For insertion of an implant into a substrate, the inserter tool 84 with the engaged implant 4b may be locked by inserting the locking pin 110 into a hole 112 or into one of a series of holes 112 in the wheel 104 configured to receive the locking pin 110. Locking the inner tool 94 and outer tool 96 together allows the implant 4b to be inserted into the substrate and prevents the insert 4b from being torqued by the inserter tool 84 before the implant 4b is in place or allows the implant to be removed from the substrate. After placing the implant 4b in the hole in the substrate, the locking pin 110 is removed from the hole 112. The knob 86 is rotated in a first direction as shown by the arrow (e.g., clockwise) while the wheel 104 is rotated in the second direction as shown by the arrow (e.g., counterclockwise). The inner tool 94 rotates the distal end of the implant in a first direction (e.g., clockwise) while the outer tool 96 rotates the proximal end of the implant in a second direction (e.g., counterclockwise). Rotating torques the implant 4b, unwinding the implant 4b, and expanding the sections in the central portion 10a of the implant 4b. By holding both ends of the implant and rotating one or both of the ends of the implant, the implant 4b need not impose torque onto the substrate. This is in contrast to a screw that is screwed into a substrate, torqueing the substrate with the screw as the screw is screwed in. The lack of torsion on a substrate with the implant disclosed herein may be useful with various substrates. It may be especially useful with smaller or weaker bones that are more sensitive to movement due to torqueing effects from screw insertion. In some variations, including with other implants, the inserter tool may be used to wind the implant around a substrate, such as when the substrate is inside the implant.

An implant may include (1) a first (proximal) end portion, (2) a second (distal) end portion (3) a central portion which (a) is a hollow body, (b) lies between, and is connected to, the first end portion and the second end portion, and (c) comprises the defined sections, which may be in either compact sections or expanded sections. The first (proximal) and second (distal) end portions and the central portion can be parts of a single unitary body (e.g., obtained by modifying a tube) or one of the end portions and the central portion can be parts of a single unitary body, and the other end portion a component that was prepared separately and then connected to the central portion; or each of the end portions can be a component that was prepared separately and then connected to the central portion. The end portions may be shaped so that they provide purchase points for torqueing and/or changing the length of the implant (fixture device). End portions include (i) those illustrated in the accompanying drawings and/or (ii) end portions having an external or internal (recessed) shape, e.g. a triangular, square or hexagonal shape.

The precursor can be converted between different configurations by one or more of a variety of different methods. A single one of the different methods can be used in a single step, or two or more of the different methods can be used in a single step or in two or more steps which can be carried out consecutively in time, or spaced apart in time. In some embodiments, before, or after, or between, one or more of the steps, part or all of the precursor can be subject to one or more heat setting steps, i.e. a step in which at least part of the precursor (in particular at least part of the defined sections) is in a desired shape, is heated to an elevated temperature, for example 500° C.-550° C., and then cooled to a lower temperature, for example by quenching in water while maintaining the precursor in the desired shape.

In one set of methods for converting the implant (precursor) between different configurations, the precursor, while the memory metal alloy is in the superelastic state, is twisted (torqued) by (a) rotating one end of the precursor without rotating the other end, or (b) rotating the ends of the precursor in different directions (at the same or different speeds), or (c) rotating the ends of the precursor in the same direction at different speeds. This makes it possible, for example, to change the configuration of the precursor (i) from a compact configuration to an expanded configuration, or (ii) from an expanded configuration to a compact configuration, or (iii) between two different compact configurations, or (iv) between two different expanded configurations. The configuration of the precursor can be changed in this way by two or more separate steps, which may be consecutive in time or spaced apart in time. In a second set of methods, while the memory metal alloy is in the superelastic state, the length of the precursor is changed. This makes it possible, for example, to change the configuration of the precursor (i) from a compact configuration to an expanded configuration, or (ii) from an expanded configuration to a compact configuration, or (iii) between two different compact configurations, or (iv) between two different expanded configurations. The configuration of the precursor can be changed in this way by two or more separate steps, which may be simultaneous, consecutive in time or spaced apart in time. The conversion of the precursor between different configurations can be accomplished by torqueing, or by changing the length of the precursor, or by a combination of torqueing and changing the length, in one or more steps which can be carried out simultaneously, consecutively in time, or spaced apart in time.

The precursor can include additional components, for example components which (a) are at the ends of the hollow body and do not include the defined sections and (b) make it easier for the hollow body to be torqued and/or changed in length and/or retained in and/or released from a delivery device. In some cases, one or more of the additional components remain in a fixture device which is ready to be delivered. In other cases, one or more of the additional components are not present in a fixture device which is ready to be delivered and/or are not present in the fixture device after it has been delivered.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant comprising:
a hollow body comprising a shape-memory material, at least part of the hollow body comprising a plurality of sections which are connected to each other and which are in either (i) a first, compact configuration in which the sections are relatively close to each other or (ii) a second, radially expanded configuration in which the sections are relatively distant from each other and longitudinally separated by an opening through a wall of the hollow body,
wherein the hollow body is configured to change between the first compact configuration and the second expanded configuration in response to an applied stimulus.

2. The implant of claim 1 wherein the sections comprise a spiral comprising shape-memory material.

3. The implant of claim 1 wherein the plurality of sections are in contact with one another when the hollow body is in the first, compact configuration.

4. The implant of claim 1 wherein the opening comprises a spiral shaped opening.

5. The implant of claim 1 wherein the hollow body comprises a cylindrical body.

6. The implant of claim 1 further comprising mechanical purchase points on the proximal and/or distal ends, wherein the implant is configured to change between the first compact configuration and the second expanded configuration in response to moving the mechanical purchase points relative to the hollow body.

7. The implant of claim 1 further comprising mechanical purchase points on the proximal and/or distal ends configured to be held by an implant inserter device.

8. The implant of claim 1 wherein the shape memory material comprises a nickel titanium alloy.

9. The implant of claim 1 wherein the shape memory material comprises a nickel titanium alloy containing about 55-56% by weight of nickel and/or about 44-45% by weight of titanium.

10. The implant of claim 1 wherein the hollow body comprises an orifice configured to pass a suture therethrough.

11. The implant of claim 1 wherein the applied stimulus comprises applying heat.

12. A method of preparing an implant comprising:
expanding a plurality of sections in a hollow body comprising of a shape-memory material, at least part of the hollow body comprising the plurality of sections which are connected to each other and which are in a first, compact configuration in which the sections are relatively close to each other to form a second, expanded configuration in which the sections are more distant from each other and separated from each other by an opening through a wall of the hollow body;
heat-setting the expanded plurality of sections with an application of heat; and
deforming the implant to convert the plurality of sections into the first, compact configuration
wherein the deformed implant comprises a shape-memory of the expanded configuration.

13. The method of claim 12 wherein expanding comprises rotating the proximal end and/or the distal end of the implant relative to a portion of the hollow body.

14. The method of 12 wherein expanding comprises rotating the proximal end of the implant in a first direction while rotating the distal end in the opposite direction.

15. The method of 12 wherein the plurality of sections are separated by a spiral cut and the expanding comprises expanding a height of the spiral cut.

16. The method of 12 wherein the plurality of sections comprise a spiral having a diameter and the expanding comprises expanding the diameter of the spiral.

17. The method of 12 further comprising modifying a proximal end and/or a distal end of the implant with mechanical purchase points.

18. A method of implanting an implant in a substrate comprising:
placing an implant comprising a proximal end, a distal end, and a hollow body therebetween into a hole in a substrate, the hollow body comprising a shape-memory material, at least part of the hollow body comprising a plurality of sections which are connected to each other and which are in a first, compact configuration in which the sections are relatively close to each other and separated by an opening through a wall of the hollow body; and
expanding with an application of heat the plurality of sections to form a second, expanded configuration in which the opening is larger and the sections are more distant from each other.

19. The method of claim 18 wherein expanding comprises rotating the proximal end and/or the distal end of the implant relative to hollow body.

20. The method of claim 18 wherein expanding comprises rotating the proximal end of the implant relative to the distal end.

21. The method of claim 18 wherein expanding comprises rotating the proximal end and distal end of the implant in opposite directions.

22. The method of claim 18 wherein expanding comprises contacting an outer surface of the wall with the substrate.

23. The method of claim 18 wherein expanding comprises contacting mechanical purchase points on the proximal end and/or distal end of the implant with an inserter device.

24. The method of claim 18 wherein the application of heat comprises heat from a mammalian body.

25. The method of claim 18 wherein the sections are between 0.5 mm and 5 mm from each other in the second, expanded configuration.

26. The method of claim 18 wherein the shape memory material comprises a nickel-titanium alloy.

27. The method of claim 18 wherein the shape memory material comprises a nickel titanium alloy containing about 55-56% by weight of nickel and/or about 44-45% by weight of titanium.

28. The method of claim 18 further comprising attaching a suture between the implant and a soft tissue in a patient.

29. The method of claim 18 wherein the substrate comprises a bone, the method further comprising drilling a hole in the bone.

30. A method of making an implant comprising:
making a cut through the wall of a central section of a hollow body, the wall comprising a shape-memory material; and
forming a plurality of sections which are connected to each other and separated by the cut, wherein the plurality of sections are relatively close to each other to form an implant in a first compact configuration.

31. The method of claim 30 wherein the cut is a spiral cut made in the central portion of a tube of constant annular section.

32. The method of claim 31 wherein the spiral cut comprises at least one turn.

33. The implant of claim 30 wherein the shape memory material comprises a nickel titanium alloy.

34. The implant of claim 30 wherein the shape memory material comprises a nickel titanium alloy containing about 55-56% by weight of nickel and/or about 44-45% by weight of titanium.

35. The method of claim 30 further comprising making a second cut through the wall of the central section of a hollow body and forming a second plurality of sections which are connected to each other.

36. A method of making an implant comprising:
making a first cut through the wall of a central section of a hollow body, the wall comprising a shape-memory material;
making a second cut parallel to the first cut through the wall;
removing wall material between the first cut and the second cut to form an opening; and
forming a plurality of sections which are connected to each other and separated by the cut, wherein the plurality of sections are in a second, expanded configuration in which the sections are relatively distant from each other and separated by an opening through a wall of the hollow body

37. The method of claim 36 wherein the cut is a spiral cut made in the central portion of a tube of constant annular section.

38. The method of claim 37 wherein the spiral cut comprises at least one turn.

39. The method of claim 36 further comprising making a second cut through the wall of the central section of a hollow body and forming a second plurality of sections which are connected to each other.

40. The implant of claim 36 wherein the shape memory material comprises a nickel titanium alloy.

41. The implant of claim 36 wherein the shape memory material comprises a nickel titanium alloy containing about 55-56% by weight of nickel and about 44-45% by weight of titanium.
